(19) United States
(12) Patent Application Publication
(10) Pub. No.: US 2016/0136448 A1
(43) Pub. Date: May 19, 2016

(54) METHOD FOR INCREASING BIO-DERIVED LOW MOLECULAR WEIGHT COMPOUND

(71) Applicant: HAKUJU INSTITUTE FOR HEALTH SCIENCE CO., LTD., Tokyo (JP)

(72) Inventor: Yuzo YAGI, Tochigi (JP)

(73) Assignee: HAKUJU INSTITUTE FOR HEALTH SCIENCE CO., LTD., Shibuya-ku, Tokyo (JP)

(21) Appl. No.: 14/898,370

(22) PCT Filed: Jul. 31, 2013
(86) PCT No.: PCT/JP13/70743

§ 371 Date: Dec. 14, 2015

(51) Int. Cl. A61N 1/40; A61N 1/04

(52) U.S. Cl. CPC A61N 1/40; A61N 1/0408

(57) ABSTRACT

The present invention is aimed to scientifically examine a change of a living body by exposing the living body in an electric field generated by a high voltage (such an action will be hereinafter also referred to “electric field exposure”), elucidate a detailed mechanism of action of a therapeutic effect by a potential therapeutic device, and find out its new use applications. The present invention is to provide a method for increasing a bio-derived low molecular weight compound including exposing a living body in an electric field generated by impressing a high voltage between electrodes or between an electrode and a ground; and a method for treating/improving a disease or a symptom thereof, the method including exposing a patient of a disease selected from bulimia, metabolic syndrome, obesity, diabetes, hyperlipemia, arteriosclerosis, headache, lumbago, stomachache, neuralgia, dementia, and sarcopenia in an electric field generated by impressing a high voltage of 9,000 to 18,000 V between electrodes or between an electrode and a ground.

### TECHNICAL FIELD

The present invention relates to a method for increasing a bio-derived low molecular weight compound. In more detail, the present invention relates to a method for increasing a bio-derived low molecular weight compound by exposing a living body in an electric field generated by a high voltage and increasing an endogenous lipid-derived signal molecule and the like in the living body.

### BACKGROUND ART

A medical device called a potential therapeutic device, in which a high voltage is impressed between electrodes or between an electrode and a ground to generate an electric field, and the body of a human being is exposed therein, thereby performing a therapy, has hitherto been used. Then, according to the Pharmaceutical Affairs Law of Japan, this potential therapeutic device is classified into the controlled medical devices (Class II), and with respect to products which meet the requirements for certification, remission of headache, stiff shoulder, chronic constipation, and insomnia is admitted as efficacy thereof.

Taking advantage of an article of the German medical journal stating that “there is no tubercular under high-voltage cables, and crop yields are large”, a Japanese inventor, Dr. HARA, Toshiyuki thought that “an electric field may possibly give good influences to human bodies” and developed a therapeutic device utilizing high-frequency hyper-pressure potential loading in 1928. It may be said that the thus developed therapeutic device is a prototype of this potential therapeutic device. After that, potential therapeutic devices by a low-frequency electric field have been sold from plural manufacturers (see PTLs 1 to 3).

A principle of the potential therapeutic device is considered to reside in the matter that a human body is encompassed by a high voltage, and a therapeutic effect is exhibited by a voltage difference from the outside. As its mechanism of action, there are considered stimulation on the skin or sensory receptor of body surface, action of an electric current induced into the living body, influence against the autonomic nervous system and peripheral circulatory improvement following that, influence against BDNF (brain-derived neurotrophic factor) or monoamine, and the like.

However, notwithstanding studies have been being advanced to date, any distinct mechanism of action has not been elucidated yet. In an era of international harmonization of pharmaceutical and medical devices, verification and evaluation of safety and validity according to the standards in Europe and U.S.A. are necessary, and elucidation of detailed mechanism of action is demanded.

### CITATION LIST

### Patent Literature

PTL 1: JP-A-5-123405

PTL 2: JP-A-2002-165888

PTL 3: W02006/082806

### Non Patent Literature

NPL 1: “Drug Discovery Today: Disease Mechanisms”, Vol. 7, No. 3-4 (2010), p.e175-e183

### SUMMARY OF INVENTION

### Technical Problem

In consequence, a problem of the present invention is to scientifically examine a change of a living body by exposing the living body in an electric field generated by a high voltage (such an action will be hereinafter also referred to “electric field exposure”) , elucidate a detailed mechanism of action of a therapeutic effect by a potential therapeutic device, and find out its new use applications.

### Solution to Problem

In order to solve the foregoing problem, the present inventor measured changes of bio-derived low molecular weight compounds in the living body, namely metabolic intermediates, hormones, signal molecules, secondary metabolites, and the like, before and after the electric field exposure by a potential therapeutic device while adopting a technique of metabolomic analysis. As a result, it has been found that in some low molecular weight compounds, their abundances are significantly increased by the electric field exposure.

In addition, among the bio-derived low molecular weight compounds to be increased by the method of the present invention, medium chain fatty acid ethanolamides, especially oleoylethanolamide is known to be an endogenous ligand against PPAR-α(peroxisome proliferator activated-receptor-alpha), a GPR119 receptor, a GPR 55 receptor, and a TRPV1 (transient receptor potential vanilloid-1) receptor (see NPL 1), and hence, it has also been known that by giving the electric field exposure to a patient of disease related to each of the above-described receptors, it becomes possible to treat the disease or improve a symptom related thereto.

The present invention is based on such findings and is related to a method for increasing bio-derived low molecular weight compound comprising exposing a living body in an electric field generated by impressing a high voltage between electrodes or between an electrode and a ground.

In addition, the present invention is related to a method for treating/improving a disease or a symptom thereof, the method comprising exposing a patient of a disease selected from bulimia, metabolic syndrome, obesity, diabetes, hyperlipemia, arteriosclerosis, headache, lumbago, stomachache, neuralgia, dementia, sarcopenia, rheumatoid arthritis, cerebral vascular disorder, attention deficit/hyperactivity disorder, major depressive disorder, and mild cognitive impairment in an electric field generated by impressing a high voltage of 9,000 to 18,000 V between electrodes or between an electrode and a ground.

### Advantageous Effects of Invention

According to the present invention, it is possible to increase the above-described endogenous low molecular weight compound existent in the living body by means of electric field exposure. Then, since the above-described endogenous low molecular weight compound also includes substances acting against receptors in the living body, it becomes possible to treat a disease related to such a receptor and improve a symptom thereof.

### BRIEF DESCRIPTION OF DRAWINGS

FIG. 1 is a perspective view showing an embodiment of a potential therapeutic device which is used for the method of the present invention.

FIG. 2 is a view schematically showing a relation of a potential in the case where a potential difference between a current table and a counter electrode is 9,000 V in the above-described potential therapeutic device.

FIG. 3 is a graph showing a change in an oleoylethanolamide concentration in a blood plasma before and after the electric field exposure of Example 2.

FIG. 4 is a graph showing a change in a palmitoylethanolamide concentration in a blood plasma before and after the electric field exposure of Example 2.

### DESCRIPTION OF EMBODIMENTS

The method of the present invention is related to a method of subjecting a living body of a human being or the like to electric field exposure using a potential therapeutic device or the like, thereby increasing a bio-derived low molecular weight compound.

Examples of the potential therapeutic device which is used in the method of the present invention include those described in PTLs 1 to 3 and the like. These are sold as a trade name, such as “HEALTHTRON”, from Hakuju Institute for Health Science Co., Ltd.

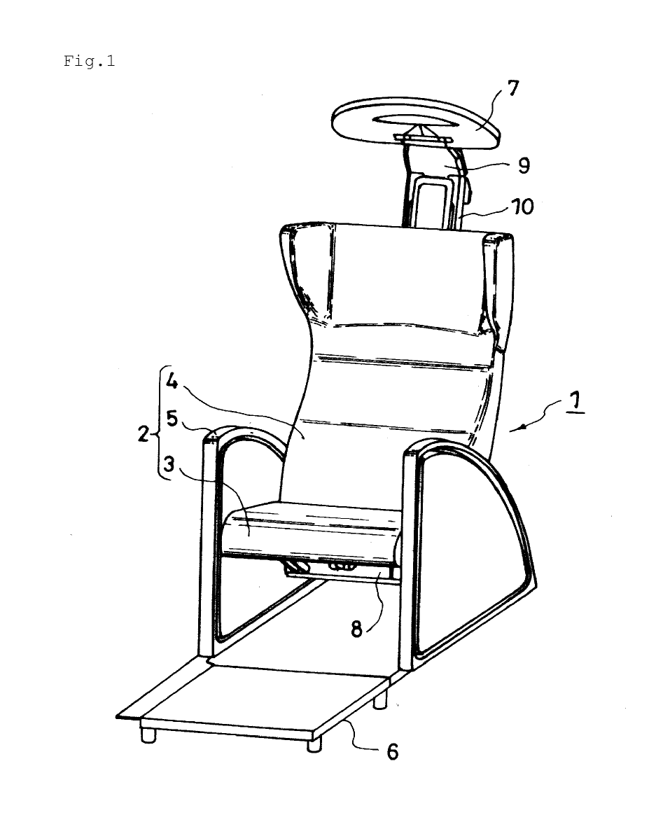

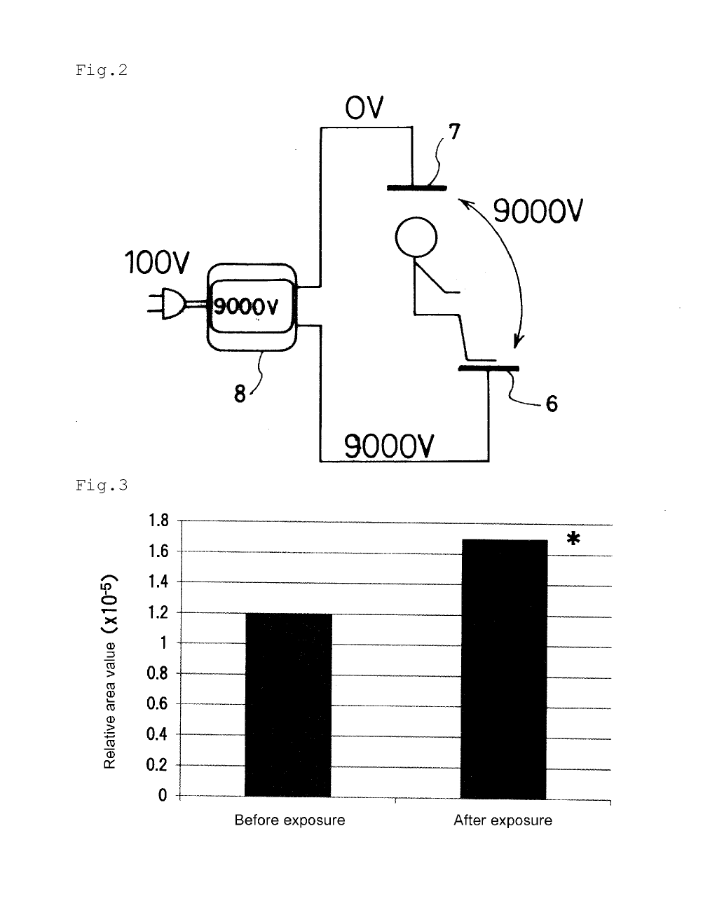

An example of this potential therapeutic device is explained by reference to FIGS. 1 and 2.

FIG. 1 is a perspective view of a potential therapeutic device, and FIG. 2 is a schematic view showing the state of a potential thereof. In the figures, 1 is a potential therapeutic device; 2 is a chair; 3 is a seat portion; 4 is a backrest portion; 5 is an armrest portion; 6 is a current table (lower electrode); 7 is a counter electrode (upper electrode); 8 is a transformer; 9 is a supporting arm; and 10 is a guide portion.

As shown in FIG. 1, the potential therapeutic device 1 which is used in the present invention is of a shape of the chair 2 including the seat portion 3, the backrest portion 4, and the armrest portion 5. Then, the current table 6 is provided at a position of feet when a person to be cured takes a seat on the chair 2, and the counter electrode 7 is provided in an extended upper portion of the backrest portion 4 of the chair 2.

Furthermore, the transform 8 is provided on the underside of the seat portion 3 of the chair 2, and a current from a commercial power supply is converted to a high voltage by this. The output side of this transformer 8 is coupled with the current table 6 and the counter electrode 7 by a code, and as shown in FIG. 2, the person to be cured who has taken a seat on the above-described chair 2 can be subjected to electric field exposure.

Although the electric field exposure in the present invention is not particularly limited in terms of a potential difference to be given, it is generally a potential difference of several hundred V to about 30,000 V, and preferably a potential difference of 9,000 to 18,000 V. In addition, though a time of the electric field exposure is not particularly limited, it is generally about 20 minutes to 60 minutes, and preferably about 30 minutes.

Incidentally, the potential therapeutic device shown in FIG. 2 is of a 2-electrode type in which the electric field is formed by the electrodes between the current table 6 on the position of feet and the counter electrode 7 over the head, it may also be of a 3-electrode type in which an electrode is further added in the seat portion 3. Moreover, it may also be of a multi-electrode type in which electrodes are added in the backrest portion 4 and the armrest portion 5.

Examples of the endogenous low molecular weight compound which can be increased by the method of the present invention include endogenous signal molecules, especially a lipid-derived signal molecule. Specifically, there can be exemplified compounds including medium chain fatty acid ethanolamides, such as oleoylethanolamide, palmitoylethanolamide, stearoylethanolamide, etc.; saturated or unsaturated fatty acids, such as oleic acid, linoleic acid, linolenic acid, arachidonic acid, cis-11-eicosenoic acid, cis-4,7,10,13,16,19-docosahexaenoic acid, cis-11,14-eicosadienoic acid, cis-8,11,14-eicosatrienoic acid, cis-5,8,11,14,17-eicosapentaenoic acid, nervonic acid, etc.; and the like.

In addition, among the above-described endogenous low molecular weight compounds, medium chain fatty acid ethanolamides, such as oleoylethanolamide, palmitoylethanolamide, etc., are large in the effects according to the method of the present invention. These have an agonist activity against PPAR-α, TRPV1, GPR119, and GPR55 receptors, and hence, by carrying out the method of the present invention, it will become possible to treat a patient of a disease in which such a receptor participates or improve a symptom thereof.

Examples of the disease to which such a receptor is related include bulimia, metabolic syndrome, obesity, diabetes, hyperlipemia, arteriosclerosis, headache, lumbago, stomachache, neuralgia, dementia, sarcopenia, rheumatoid arthritis, cerebral vascular disorder, attention deficit/hyperactivity disorder, major depressive disorder, mild cognitive impairment, and the like, and the following effects targeting these diseases are expected.

(1) Pain alleviation and analgesia effect

(2) Functional dyspepsia (e.g., gastric hyperesthesia and abnormal intestinal movement)

(3) Sarcopenia/amyotrophy

(4) Eating disorder and diet generated from appetite suppression

(5) Lipid improvement measure against metabolic syndrome or obesity

(6) Hyperlipemia, coronary artery disease, improvement of insulin resistance, and diabetes

(7) Improvement of memory/learning function

(8) Anti-inflammatory effect

### EXAMPLES

Next, the present invention is hereunder described in more detail by reference to Examples, but it should be construed that the present invention is not limited to these Examples at all.

### Example 1

An electric field exposure test was performed with respect to ten healthy subjects (age: 40.2±10.2 years, BMI: 22.0±2.4). For this test, HEALTHTRON PRO-18T (manufactured by Hakuju Institute for Health Science Co., Ltd., Tokyo, Japan) was used, a potential difference between the current table and the counter electrode was set to 18,000 V, and this state was kept for 30 minutes.

Before and after the electric field exposure test, a blood sample was collected from each of the subjects by using an EDTA blood collection tube (a product name: VP-NA070K, manufactured by Terumo Corporation, Tokyo, Japan) and immediately subjected to centrifugation at 800×g for 5 minutes, thereby separating a blood plasma from other cellular materials. Subsequently, the resulting blood plasma was transferred into an unused Eppendorf type tube and stored at −80° C. before the treatment, thereby preparing a sample for metabolomic analysis before and after the electric field exposure.

500 μL of the resulting plasma sample was added to 1,500 μL of an internal standard solution containing 1% of a formic acid/acetonitrile mixed solution (solution ID: H3304-1002, manufactured by Human Metabolome Technologies Inc., Tsuruoka-city, Japan) at 0° C. to inactivate oxygen, and the resultant was thoroughly stirred and subjected to centrifugation at 2,300×g at 4° C. for 5 minutes, thereby removing a solid.

Subsequently, a supernatant was filtered by using Hybrid SPE phospholipid 55261-U (manufactured by Supelco Inc., Bellefonte, Pa., U.S.A.), thereby removing a phospholipid. The resulting filtrate was dried and then dissolved with 100 μL of an isopropanol/Milli-Q mixed solution and subjected to an LC-TOFMS analysis in the following manner.

For the LC-TOFMS analysis, an Agilent LC system (a product name: Agilent 1200 series RRLC system SL, manufactured by Agilent Technologies, Inc., Waldbronn, Germany) equipped with an Agilent 6230 time-of-flight mass spectrometer (manufactured by Agilent Technologies, Inc., Waldbronn, Germany) was used, and an ODS column (2×50 mm, 2 was used as a column.

For the control of the system to be used for the LC-TOFMS analysis, Agilent G2201AA ChemStation software version B.03.01, manufactured by Agilent Technologies, Inc., Waldbronn, Germany was used.

In the above-described analysis, a cationic compound and an ionic compound were analyzed by an already-known method *1. In addition, a peak was extracted using an automated, integrated software (a product name: MasterHands, manufactured by Keio University, Tsuruoka-city, Japan), and information regarding the peak, such as an m/z value, a holding time of the LC-TOFMS analysis, a peak area value, etc., was obtained from an already-known literature *2.

*1: Ooga, T. et al., “Metabolomic anatomy of an animal model revealing homeo-static imbalances in dyslipidaemia”, Mol. Biosyst., 7, 1217-1223, 2011

*2: Sugimoto, M. et al., “Capillary electrophoresis mass spectrometry-based saliva metabolomics identified oral, breast and pancreatic cancer-specific profiles”, Metabolomics, 6, 78-95, 2010

In the analysis, peak signals corresponding to isotopomers, adduct ions, and other produced ions of known metabolites were excluded, and peaks other than these were subjected to peak annotation with a putative metabolite of an HMT metabolite data base on the basis of measurement time/holding time and m/z values obtained by the TOFMS analysis. An error of the peak annotation was set to ±5 minutes for the measurement time and ±10 ppm for the m/z value, respectively.

### [Results]

A peak detected by the above-described method was 278. Among those, the results regarding the majority are shown in Table 1.

TABLE 1

| | Relative area value | | Relative ratio | |
|---|---|---|---|---|
| | Before exposure | After exposure | [(After exposure)/ | |
| Compound name | (Mean ± SD) | (Mean ± SD) | (Before exposure)] | P value |
| cis-4,7,10,13,16,19- | 3.8E−04 ± 2.9E−04 | 5.8E−04 ± 4.1E−04 | 1.53 | 0.003 ** |
| Docosahexaenoic acid | | | | |
| Oleic acid | 1.2E−03 ± 7.9E−04 | 1.8E−03 ± 1.1E−03 | 1.50 | 0.009 ** |
| Linoleic acid | 4.7E−04 ± 2.5E−04 | 6.9E−04 ± 3.7E−04 | 1.47 | 0.017 * |
| cis-11,14- | 8.9E−06 ± 5.4E−06 | 1.3E−05 ± 8.1E−06 | 1.46 | 0.007 ** |
| Eicosadienoic acid | | | | |
| cis-11-Eicosenoic acid | 1.5E−05 ± 9.5E−06 | 2.1E−05 ± 1.5E−05 | 1.40 | 0.014 * |
| Arachidonic acid | 1.0E−04 ± 4.4E−05 | 1.4E−04 ± 7.1E−05 | 1.40 | 0.004 ** |
| cis-5,8,11,14,17- | 3.2E−05 ± 2.2E−05 | 4.4E−05 ± 3.0E−05 | 1.38 | 0.004 ** |
| Eicosapentaenoic acid | | | | |
| cis-8,11,14- | 1.1E−05 ± 3.8E−06 | 1.5E−05 ± 6.0E−06 | 1.36 | 0.002 ** |
| Eicosatrienoic acid | | | | |
| Palmitic acid | 2.5E−04 ± 1.3E−04 | 3.4E−04 ± 1.8E−04 | 1.36 | 0.019 * |
| Linolenic acid | 4.6E−05 ± 2.6E−05 | 6.2E−05 ± 3.2E−05 | 1.35 | 0.044 * |
| Oleoylethanolamide | 2.2E−05 ± 5.1E−06 | 2.7E−05 ± 8.4E−06 | 1.23 | 0.009 ** |
| Palmitoylethanolamide | 2.1E−05 ± 4.0E−06 | 2.4E−05 ± 5.3E−06 | 1.14 | 0.017 * |

As a result, before and after the electric field exposure in the above-described test, a significant increase at a significance level of 1% was observed in oleic acid, cis-4,7,10,13,16,19-docosahexaenoic acid, cis-11,14-eicosadienoic acid, stearic acid, arachidonic acid, heptadecanoic acid, 2-hydroxybutyric acid, cis-8,11,14-eicosatrienoic acid, FA (17:0), cis-5,8,11,14,17-eicosapentaenoic acid, nervonic acid, oleoylethanolamide, and dehydroisoandrosterone-3-sulfate; and a significant increase at a significant level of 5% was observed in linoleic acid, FA (22:5), FA (22:4), cis-11-eicosenoic acid, ethyl arachidonate, palmitic acid, FA (19:1), linolenic acid, UDP, FA (14:1), phosphocreatine, threonic acid T, N-acetylalanine, N-acetylglycine, hecogenin, stearoylethanolamide, palmitoylethanolamide, isethionic acid, and etiocholane-3α-ol-17-one sulfate.

### Example 2

An electric field exposure test was performed with respect to five healthy subjects (age: 42.8±10.1 years, BMI: 21.8±1.9) . For this test, HEALTHTRON Hb9000T (manufactured by Hakuju Institute for Health Science Co., Ltd., Tokyo, Japan) was used, a potential difference between the current table and the counter electrode was set to 9,000 V, and this state was kept for 30 minutes.

[Results]

A peak detected in the same method as in Example 1 was 271. Among those, the results regarding the majority are shown in Table 2.

TABLE 2

| | Relative area value | | Relative ratio |
|---|---|---|---|
| | Before exposure | After exposure | [(After exposure)/ |
| Compound name | (Mean ± SD) | (Mean ± SD) | (Before exposure)] |
| cis-4,7,10,13,16,19- | 1.2E−03 ± 6.9E−04 | 1.8E−03 ± 9.4E−04 | 1.50 |
| Docosahexaenoic acid | | | |
| Oleic acid | 8.3E−04 ± 3.9E−04 | 1.4E−03 ± 6.1E−04 | 1.69 |
| Linoleic acid | 2.0E−03 ± 8.7E−04 | 3.1E−03 ± 1.3E−03 | 1.55 |
| cis-11,14- | 3.4E−05 ± 1.7E−05 | 5.5E−05 ± 2.2E−05 | 1.62 |
| Eicosadienoic acid | | | |
| cis-11-Eicosenoic acid | 5.9E−05 ± 4.6E−05 | 9.2E−05 ± 5.2E−05 | 1.56 |
| Arachidonic acid | 2.6E−04 ± 1.2E−04 | 3.9E−04 ± 1.6E−04 | 1.50 |
| cis-5,8,11,14,17- | 6.5E−05 ± 4.1E−05 | 1.0E−04 ± 6.2E−05 | 1.54 |
| Eicosapentaenoic acid | | | |
| cis-8,11,14- | 3.4E−05 ± 1.4E−05 | 4.9E−05 ± 1.7E−05 | 1.44 |
| Eicosatrienoic acid | | | |
| Palmitic acid | 1.4E−03 ± 4.4E−04 | 2.1E−03 ± 7.4E−04 | 1.50 |
| Linolenic acid | 1.6E−04 ± 8.8E−05 | 2.8E−04 ± 1.2E−04 | 1.75 |
| Oleoylethanolamide | 1.2E−05 ± 3.3E−06 | 1.7E−05 ± 5.4E−06 | 1.42 |
| Palmitoylethanolamide | 1.8E−05 ± 4.0E−06 | 2.3E−05 ± 4.7E−06 | 1.28 |

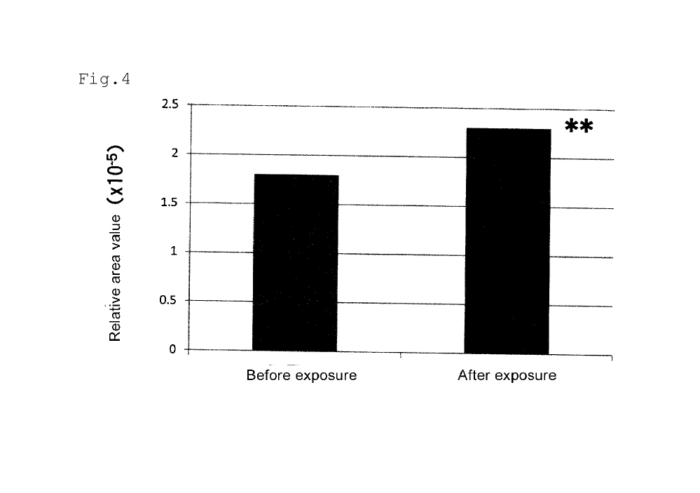

Changes in plasma concentrations of oleoylethanolamide and palmitoylethanolamide before and after the electric field exposure in this Example are shown in FIGS. 3 and 4, respectively.

### INDUSTRIAL APPLICABILITY

According to the method of the present invention, endogenous low molecular weight compounds can be increased by a simple means of exposing a living body in an electric field generated by a high voltage. In consequence, by carrying out the method of the present invention, it becomes possible to treat diseases in which such endogenous low molecular weight compound and receptor participate, for example, bulimia, metabolic syndrome, obesity, diabetes, hyperlipemia, arteriosclerosis, headache, lumbago, stomachache, neuralgia, dementia, sarcopenia, rheumatoid arthritis, cerebral vascular disorder, attention deficit/hyperactivity disorder, major depressive disorder, mild cognitive impairment, etc., and also to improve symptoms by these diseases. Thus, the method of the present invention can be utilized as a new therapeutic method.

### REFERENCE SIGNS LIST

1: Potential therapeutic device

2: Chair

3: Seat portion

4: Backrest portion

5: Armrest portion

6: Current table (lower electrode)

7: Counter electrode (upper electrode)

8: Transformer

9: Supporting arm

10: Guide portion

1. A method for increasing an amount of at least one bio-derived compound in a living body comprising:
exposing the living body in an electric field generated between electrodes or between an electrode and a ground for a time sufficient to increase an amount of said at least once bio-derived compound compared to an amount prior to exposure to the electric field.

2. The method for increasing a bio-derived low molecular weight compound according to claim 1, wherein a potential difference in the electric field is 9,000 to 18,000 V.

3. The method for increasing a bio-derived low molecular weight compound according to claim 1, wherein a time of exposing the living body in the electric field is 20 to 60 minutes.

4. The method for increasing a bio-derived low molecular weight compound according to claim 1, wherein the bio-derived low molecular weight compound is a lipid-derived signal molecule.

5. The method for increasing bio-derived low molecular weight compound according to claim 1, wherein the bio-derived low molecular weight compound is a medium chain fatty acid ethanolamide or a saturated or unsaturated fatty acid.

6. A method for reducing the severity of a disease or a symptom thereof, comprising exposing a living body in an electric field generated between electrodes or between an electrode and a ground, wherein a potential difference in the electric field is 9,000 to 18,000 V.

7. The method for reducing the severity of a disease or a symptom thereof according to claim 6, wherein the living body is exposed to the electric field for 20 to 60 minutes.

8. The method of claim 6, wherein said living body is a patient having a disease selected from bulimia, metabolic syndrome, obesity, diabetes, hyperlipemia, arteriosclerosis, headache, lumbago, stomachache, neuralgia, sarcopenia, rheumatoid arthritis, and cerebral vascular disorder.

9. The method of claim 6, wherein said living body is a patient having a disease selected from the group consisting of dementia, attention deficit/hyperactivity disorder, major depressive disorder, and mild cognitive impairment.
